(19) United States
(12) Patent Application Publication
(10) Pub. No.: US 2026/0306408 A1
(43) Pub. Date: Oct. 8, 2026

(54) SUBCUTANEOUS PEPTIDE DELIVERY SYSTEM

(71) Applicant: William Demarest Johnson, Los Angeles, CA (US)

(72) Inventor: William Demarest Johnson, Los Angeles, CA (US)

(21) Appl. No.: 19/170,465

(22) Filed: Apr. 4, 2025

(51) Int. Cl. A61K 9/16; A61K 9/00; A61K 38/10; A61K 38/12

(52) U.S. Cl. CPC A61K 9/1635; A61K 9/0024; A61K 38/10; A61K 38/12

(57) ABSTRACT

A method of administering a subcutaneous peptide delivery system may include administering 0.04 mg per day via single subcutaneous cannula insertion of a subcutaneous peptide delivery system including approximately 90% peptide and an inert conveyer.

### TECHNICAL FIELD

The embodiments generally relate to the technical field of systems and methods for subcutaneous peptide delivery.

### BACKGROUND

Conventional methods of administering therapeutic peptides primarily rely on subcutaneous injections, which require frequent administration and can lead to issues with patient compliance. Daily injections may cause discomfort, inconvenience, and an increased risk of infection, particularly for individuals requiring long-term peptide therapy. Additionally, maintaining a steady therapeutic level of peptides in the bloodstream is challenging due to fluctuations in absorption rates, leading to inconsistent efficacy.

While some drug delivery technologies, such as transdermal patches, controlled-release capsules, and infusion pumps, have been developed to provide sustained drug release, these methods are largely designed for small-molecule drugs and hormones. Non-hormonal peptides present additional challenges due to their inherent instability, susceptibility to enzymatic degradation, and difficulty in achieving controlled, long-term release without degradation. Prior sustained-release implants have primarily been limited to hormone-based therapies, leaving a gap in the field for non-hormonal peptide delivery solutions.

Existing subcutaneous drug delivery implants typically rely on polymeric carriers to regulate release kinetics. However, these systems often require complex formulations, biocompatibility testing, and long regulatory approval processes. Furthermore, many of these implants provide zero-order release, which may not be optimal for certain peptides requiring gradual and continuous absorption. A need exists for a simple, efficient, and minimally invasive method to deliver non-hormonal peptides over extended periods while maintaining peptide stability and ensuring patient adherence.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

A subcutaneous peptide delivery system provides a sustained and controlled release of non-hormonal peptides over an extended period, eliminating the need for frequent injections. The system comprises a pellet containing approximately 90% peptide and an inert carrier, such as Polivinilpirrolidon (PVP K-90), designed to maintain stability and control the peptide's release rate. Once inserted into the subcutaneous tissue via a small cannula, the pellet gradually dissolves, delivering a precise daily dosage of 0.04 mg over a three-month period.

The system accommodates various peptide formulations, including bremelanotide (PT-141), gastric pentadecapeptide (BPC-157), and MOTs-C, enabling its application in a range of therapeutic areas. The pellet's dimensions, approximately 3 mm in length and 2 mm in diameter, facilitate minimally invasive administration and ensure patient comfort. By utilizing an inert carrier, the system stabilizes the peptides in solid form, reducing degradation and enhancing bioavailability.

Unlike conventional polymer-based drug delivery implants, which often require complex formulations and rigid zero-order release kinetics, the disclosed system offers a simpler approach with predictable, steady absorption. This design improves patient adherence by removing the burden of daily injections while maintaining consistent therapeutic levels of the peptide. Additionally, the subcutaneous pellet eliminates the need for external infusion devices, reducing patient discomfort and the risk of compliance failure.

The system is designed to be biocompatible and biodegradable, allowing the pellet to dissolve naturally within the subcutaneous tissue without requiring removal. The controlled-release matrix ensures the peptide remains effective throughout the three-month period, optimizing therapeutic outcomes while minimizing side effects. The pellet composition and release kinetics can be tailored to specific peptides, expanding its applicability across multiple treatment areas.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a method of using a subcutaneous peptide delivery system, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The subcutaneous peptide delivery system comprises a solid pellet formed from approximately 90% of a specified peptide and an inert carrier. The inert carrier is selected to provide structural integrity and controlled release properties, ensuring a sustained dosage over an extended period. In one embodiment, the carrier is Polivinilpirrolidon (PVP K-90), which has been demonstrated to provide stability and controlled dissolution of the peptide.

The pellet is cylindrical, with a length of approximately 3 mm and a diameter of approximately 2 mm. This size allows for minimally invasive insertion through a small-gauge cannula, reducing patient discomfort and facilitating ease of administration. Once inserted into the subcutaneous tissue, the pellet remains in place, where it gradually dissolves and releases the peptide into the bloodstream at a controlled rate.

The controlled-release mechanism of the pellet is designed to provide a steady peptide dosage of approximately 0.04 mg per day in vitro. This gradual release maintains stable peptide levels in the body, reducing fluctuations commonly associated with traditional injection-based peptide therapy. The release rate is influenced by the composition of the pellet, including the ratio of peptide to inert carrier and the solubility characteristics of the peptide itself.

The system supports multiple peptide formulations, including but not limited to bremelanotide (PT-141), gastric pentadecapeptide (BPC-157), and MOTs-C. Each peptide is incorporated into the pellet with the appropriate inert carrier to ensure stability and controlled release. The solid-state form of the peptide within the pellet minimizes degradation, allowing for long-term storage and reliable administration.

In one embodiment, the pellet is introduced into the subcutaneous tissue through a specialized cannula, which is designed to minimize tissue trauma and ensure precise placement. Once inserted, the pellet does not require removal, as it gradually dissolves over a period of approximately three months. The biocompatible composition of the pellet ensures that there is no adverse immune response or need for additional intervention.

The pellet-based delivery system offers significant advantages over existing peptide administration methods. Traditional subcutaneous injections require daily or frequent administration, which can be inconvenient and painful for patients. The disclosed system eliminates the need for frequent injections, improving patient compliance and treatment adherence. Additionally, the controlled release of the peptide minimizes peak-and-trough fluctuations, which can improve therapeutic outcomes and reduce side effects.

Unlike polymer-based drug delivery implants, which often require complex formulation strategies to regulate drug release, the disclosed system utilizes a simpler approach based on peptide composition and an inert carrier. The absence of external infusion devices or reservoirs makes the system more user-friendly and less prone to mechanical failures.

The release kinetics of the pellet can be modified by adjusting the peptide-to-carrier ratio and selecting alternative carriers with different dissolution profiles. This adaptability enables the system to be tailored to different peptides and therapeutic applications, making it a versatile platform for sustained peptide delivery.

In some embodiments, additional modifications to the pellet composition may be made to enhance its biocompatibility and dissolution characteristics. Coatings or encapsulating agents may be applied to further regulate the release rate and provide additional protection against enzymatic degradation. However, the base composition of the system remains focused on delivering a stable, continuous dose of peptide without requiring patient intervention.

The subcutaneous peptide delivery system addresses a long-standing need for a convenient, effective, and reliable method of administering non-hormonal peptides. By providing a sustained-release pellet with optimized stability and biocompatibility, the system enhances patient adherence, reduces treatment burden, and improves therapeutic efficacy.

The disclosed system is applicable across a variety of medical fields, including regenerative medicine, metabolic disorders, and immune system modulation. By enabling controlled, long-term peptide release, the system supports more consistent treatment outcomes and enhances patient quality of life. The flexibility of the system to accommodate different peptides and release rates further underscores its potential as a transformative approach to peptide therapy.

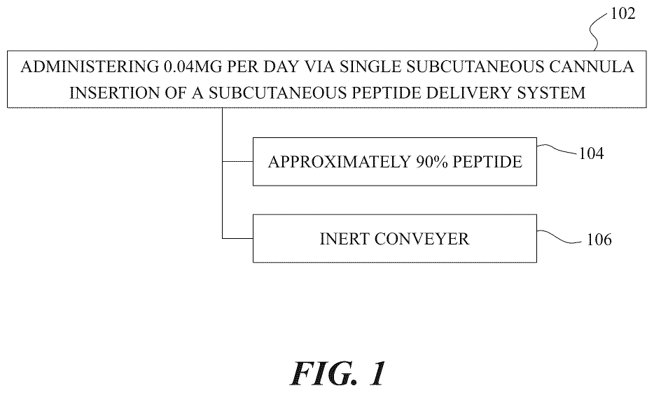

FIG. 1 depicts a variation of the disclosed method of administering a subcutaneous peptide delivery system. In step 102, the method may include administering 0.04 mg per day via single subcutaneous cannula insertion of a subcutaneous peptide delivery system including approximately 90% peptide 104 and an inert conveyer 106.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A subcutaneous peptide delivery system, comprising:
approximately 90% peptide; and
an inert conveyer.

2. The subcutaneous peptide delivery system of claim 1, wherein the subcutaneous peptide delivery system is a pellet.

3. The subcutaneous peptide delivery system of claim 1, wherein the pellet is approximately 3 mm in length and is approximately 2 mm in diameter.

4. The subcutaneous peptide delivery system of claim 1, wherein the peptide is a non-hormonal peptide.

5. The subcutaneous peptide delivery system of claim 1, wherein the peptide comprises bremelanotide (PT-141).

6. The subcutaneous peptide delivery system of claim 1, wherein the peptide comprises gastric pentadecapeptide (BPC-157).

7. The subcutaneous peptide delivery system of claim 1, wherein the peptide comprises MOTs-C.

8. The subcutaneous peptide delivery system of claim 1, wherein the inert conveyer comprises Polivinilpirrolidon (PVP K-90).

9. The subcutaneous peptide delivery system of claim 1, wherein the subcutaneous peptide delivery system comprises a controlled release rate of 0.04 mg per day in vitro.

10. A method of administering a subcutaneous peptide delivery system, comprising:
administering 0.04mg per day via single subcutaneous cannula insertion of a subcutaneous peptide delivery system comprising approximately 90% peptide and an inert conveyer.

11. The method of claim 10, wherein the subcutaneous peptide delivery system is a pellet.

12. The method of claim 10, wherein the pellet is approximately 3 mm in length and is approximately 2 mm in diameter.

13. The method of claim 10, wherein the peptide is a non-hormonal peptide.

14. The method of claim 10, wherein the peptide comprises bremelanotide (PT-141).

15. The method of claim 10, wherein the peptide comprises gastric pentadecapeptide (BPC-157).

16. The method of claim 10, wherein the peptide comprises MOTs-C.

17. The method of claim 10, wherein the inert conveyer comprises Polivinilpirrolidon (PVP K-90).

18. The method of claim 10, wherein the subcutaneous peptide delivery system comprises a controlled release rate of 0.04 mg per day in vitro.

19. The method of claim 10, wherein the administering occurs consecutively over three months.
